(19) United States
(12) Patent Application Publication
(10) Pub. No.: US 2025/0179552 A1
(43) Pub. Date: Jun. 5, 2025

(54) TRIPLET-TRIPLET ANNIHILATION UPCONVERSION NANOPARTICLES BASED SELF-STANDING BIOLOGICAL SENSORS, AND DEVICES AND METHODS THEREOF

(71) Applicant: University of Massachusetts, Westborough, MA (US)

(72) Inventors: Gang Han, Shrewsbury, MA (US); Ling Huang, Worcester, MA (US)

(21) Appl. No.: 18/844,462

(22) PCT Filed: Mar. 7, 2023
(86) PCT No.: PCT/US23/14749

§ 371 Date: Sep. 6, 2024

Related U.S. Application Data
(60) Provisional application No. 63/320,063, filed on Mar. 15, 2022.

(51) Int. Cl. C12Q 1/26; C09K 11/02; C09K 11/06; C12Q 1/54; G01N 33/543

(52) U.S. Cl. CPC C12Q 1/26; C09K 11/025; C09K 11/06; C12Q 1/54; G01N 33/54346; C09K 2211/1011; C09K 2211/1029; C09K 2211/185; G01N 2333/904

(57) ABSTRACT

The invention provides novel triplet-triplet annihilation upconversion nanoparticles as background free self-standing biological sensors, and devices and methods thereof.

### PRIORITY CLAIMS AND RELATED APPLICATIONS

This application claims the benefit of priority to U.S. Provisional Application No. 63/320,063, filed Mar. 15, 2022, the entire content of which is incorporated herein by reference for all purposes.

### TECHNICAL FIELDS OF THE INVENTION

The invention generally relates to upconversion nanoparticles and their use as biological sensors. More particularly, the invention provides triplet-triplet annihilation upconversion nanoparticles as background free self-standing biological sensors, and devices and methods thereof.

### BACKGROUND OF THE INVENTION

Triplet-triplet annihilation upconversion nanoparticle (TTA-UCNP) has been emerging as the next generation of photon upconversion materials. These nanoparticles hold great promise in a wide variety of important areas, such as bioimaging, therapy, photoredox catalysis and solar energy harvesting, because of their unique properties such as high upconversion quantum yield, the need for low excitation power long wavelength light (<200 mW/cm²) and potentially tunable excitation/emission wavelength. In particular, optical triplet-triplet annihilation upconversion (TTA-UC) converts low-energy long wavelength photon to high-energy shorter wavelength one. During the process of TTA-UC, low energy photons are first absorbed by a photosensitizer (Sen), which then reach its single excited state (¹Sen*). Subsequently, the intersystem crossing (ISC) process occurs. The energy of the photosensitizers transited to their triplet excited state (³Sen*). Due to the long-lived lifetime of triplet excited state, the energy of ³Sen* can transfer to annihilator (An) by triplet-triplet energy transfer process (TTET). Finally, two triplet excited state annihilators (³An*) undergo a TTA process to generate one high-energy singlet excited state annihilator (¹An*), and subsequent short-wavelength light emissions. (Singh-Rachford, et al. 2010 Coord. Chem. Rev. 254, 2560-2573; Zhou, et al. 2015 Chem. Rev. 115, 395-465; Zhao, et al. 2011 RSC Adv. 1, 937-950; Filatov, et al. 2016 Chem. Soc. Rev. 45, 4668-4689; Zhao, et al. 2015 Chem. Soc. Rev. 44, 8904-8939; Zhao, et al. 2013 Chem. Soc. Rev. 42, 5323-5351; Zhu, et al. 2017 Chem. Soc. Rev. 46, 1025-1039; Peng, et al. 2015 Chem. Rev. 115, 7502-7542; Liu, et al. 2012 J. Am. Chem. Soc. 134, 5390-5397; Kwon, et al. 2016 ACS Nano. 10, 1512-1521; Huang, et al. 2017 Angew. Chem. Int. Ed. 56, 14400-14404; Ravetz, et al. 2019 Nature. 565, 343-346; Schulze, et al. 2015 Energy Environ. Sci. 8, 103-125.)

To date, a few TTA-UC photosensitizers and annihilators pairs with solid upconversion quantum yields have been reported in deaerated organic solvents. Unfortunately, oxygen molecules diffused in water can rapidly quench ³Sen* and ³An*. In particular, ³Sen* can rapidly sensitize oxygen molecules to generate singlet oxygen (¹O₂), thereby inhibiting the TTET process from ³Sen* to ³An*, leading to the failure of TTA-UC (FIG. 1a, left portion). Therefore, although TTA-UC can be improved in organic solvent by the approaches such as oxygen consuming, enhancing upconversion efficiency of TTA-UCNP in aqueous solutions has been challenging under an ambient atmosphere. (Zhao, et al. 2011 RSC Adv. 1, 937-950; Singh-Rachford, et al. 2008 J. Am. Chem. Soc. 130, 16164-16165; Singh-Rachford, et al. 2009 J. Am. Chem. Soc. 131, 12007-12014.)

This oxygen-induced quenching problem is an especially strong obstacle in regard to biological applications that must happen in aqueous solutions under an ambient atmosphere. In order to address this key problem, several methods have been attempted in recent years. For example, TTA-UC dyes were encapsulated in mesoporous silica to slow down oxygen diffusion and the concomitant oxygen quenching. However, the existing silica TTA-UCNP cannot block the oxygen quenching completely and thus has suboptimal upconversion performance. In addition, the size of the nanoparticles is very large, typically over 200 nm. Liposomes were also used to encapsulate TTA-UC dyes in order to construct water-dispersible nanoparticles. Similarly, this method was also unable to effectively resolve the oxygen issue in aqueous solution. Recently, to reduce the oxygen-induced TTA-UC quenching, soybean oil has been explored to form TTA-UC oil droplets. Yet, because of the quick oil/water phase separation, the resultant TTA-UC oil droplet appears to have difficulty staying in aqueous solution. In addition, these TTA-UC oil droplets are also large, typically over 100 nm. (Kwon, et al. 2016 ACS Nano. 10, 1512-1521; Huang, et al. 2019 Biomaterials. 201, 77-86; Huang, et al. 2018 Small Methods. 2, 1700370; Askes, et al. 2018 Nat. Rev. Chem. 2, 437-452; Kwon, et al. 2015 ACS Appl. Mater. Interfaces. 7, 318-325; Askes, et al. 2014 Angew. Chem. Int. Ed. 53, 1029-1033; Baluschev, et al. 2016 Mater. Horiz. 3, 478-486; Kouno, et al. 2016 Chem. Sci. 7, 5224-5229; Liu, et al. 2013 J. Am. Chem. Soc. 135, 5029-5037; Tian, et al. 2017 Biomaterials. 112,10-19; Xu, et al. 2018 Nat. Commun. 9, 2698.)

Therefore, a simple strategy to overcome oxygen-quenching trouble for stable water-dispersible and small-size TTA-UCNP under an ambient atmosphere is both urgently needed.

### SUMMARY OF THE INVENTION

The invention provides novel and simple enzymatic strategy to overcome oxygen-induced triplet-triplet annihilation upconversion quenching. The disclosed invention addresses the long-felt challenges in aqueous TTA-UC due to its sensitivity to oxygen, which has hindered TTA-UC's biological applications under ambient atmosphere. This novel strategy stems from glucose oxidase catalyzed glucose oxidation reaction, which enables rapid oxygen depletion to turn on upconversion in the aqueous solution. Furthermore, self-standing upconversion biological sensors of such nanoparticles are developed to detect glucose and measure the activity of enzymes related to glucose metabolism in a highly specific, sensitive, and background-free manner. The disclosed invention not only overcomes the key roadblock for applications of triplet-triplet annihilation upconversion nanoparticles in aqueous solutions, it also enables the development of triplet-triplet annihilation upconversion nanoparticles as background free self-standing biological sensors.

In one aspect, the invention generally relates to an upconversion nanoparticle comprising an organic photosensitizer and an organic annihilator pair encapsulated in a polymeric material, wherein upon excitation the organic photosensitizer-annihilator pair is capable of undergoing annihilation upconversion to generate emission having a shorter wavelength than that of the excitation.

In another aspect, the invention generally relates to an aqueous composition comprising a plurality of the upconversion nanoparticle disclosed herein.

In yet another aspect, the invention generally relates to a sensor device that comprises upconversion nanoparticles or an aqueous composition disclosed herein.

In yet another aspect, the invention generally relates to a method for detecting glucose. The method comprises: providing an aqueous composition comprising upconversion nanoparticles disclosed herein and glucose oxidase; measuring a rate of triplet-triplet annihilation upconversion; adding a test sample to the aqueous composition; measuring the rate of triplet-triplet annihilation upconversion again; and analyzing the presence and/or concentration of glucose based on measurements with and without the test sample.

In yet another aspect, the invention generally relates to a method for measuring activity of an enzyme related to glucose metabolism. The method comprises: providing an aqueous composition comprising upconversion nanoparticles disclosed herein and glucose; measuring a rate of triplet-triplet annihilation upconversion; adding to the aqueous composition a test sample;

measuring the rate of triplet-triplet annihilation upconversion again; and analyzing enzymatic activity based on measurements with and without the test sample.

### BRIEF DESCRIPTION OF THE DRAWINGS

FIG. 1. Solving the oxygen-quenching issue of TTA-UCNP with glucose oxidase. a) Schematic illustration and the mechanism of lighting up TTA-UCNP in the presence of glucose and glucose oxidase (GOX). Blue dot represents the annihilator of perylene, the red dot represents the photosensitizer of PdTPBP, the left grey nanoparticles stand for origin TTA-UCNP and the right green nanoparticles stands for TTA-UCNP in the presence of GOX and glucose. b) Molecular structures of the photosensitizer PdTPBP, the annihilator perylene and the amphiphilic polymer PAA-OA.

FIG. 2. Upconversion performance of TTA-UCNP in different circumstance. a) TTA-upconversion spectra of TTA-UCNP at different conditions (TTA-UCNP only, TTA-UCNP with glucose, or with GOX, or with both glucose and GOX) in PBS buffer, λₑₓ=650 nm. b) Incident light power dependence study of TTA-upconversion emission intensity for TTA-UCNP in PBS buffer. c) Double-logarithmic plot of perylene integrated emission intensity as a function of 650 nm excitation power density. Solid lines illustrate a slope of 2.05 (black, quadratic) and a slope of 1.07 (red, linear), Iₜₕ is 138.9 mW cm⁻². d) The upconversion quantum yield and upconversion brightness of TTA-UCNP with different 650 nm incident power density. c(glucose)=5 mg mL⁻¹, c(GOX)=32.5 μg mL⁻¹.

FIG. 3. TTA-UNCP for efficient sensing of glucose. a) The response times of the mixture of TTA-UCNP (1 mg mL⁻¹) and GOX (10 μg mL⁻¹) with different concentrations of glucose, λₑₓ=650 nm, 100 mW cm⁻². b) In-In coordination fitting of glucose concentration and half response times in deionized water, slope=−0.62783, R=0.9995, n=3 means that each experiment is repeated three times independently, the error bar represents the mean of the three times±standard deviation (SD). c) The TTA-UCNP in the cell medium of 20% FBS, phenol red and GOX (10 μg mL⁻¹) in the absence (left) and in the presence (right) of light, λₑₓ=650 nm. d) In-In coordination fitting of glucose concentration and half response times of TTA-upconversion in TTA-UCNP in cell culture medium including 20% FBS, phenol red and GOX (10 μg mL⁻¹), slope=−0.6333, R=0.994, n=3 means that each experiment is repeated three times independently, the error bar represents the mean of the three times±standard deviation (SD).

FIG. 4. TTA-UCNP measures the activity of invertase. a) Schematic illustration of the mechanism of TTA-UCNP as biosensor for invertase enzymatic reactions. b) The invertase concentration dependent TTA-upconversion turn-on response. λₑₓ=650 nm, 100 mW cm⁻². c) In-In coordination fitted linear relationship between the concentration of invertase and TTA-upconversion half response time (slope=−0.625, R=0.999), n=3 means that each experiment is repeated three times independently, the error bar represents the mean of the three times±standard deviation (SD).

FIG. 5. Normalized absorption and emission spectra of PdTPBP and perylene in degassed toluene.

FIG. 6. The perylene-concentration dependent upconversion. a) Upconversion emission spectra of mixed solutions of PdTPBP (10 μM) with different concentrations of perylene in degassed toluene. b) The respective integrated TTA upconversion intensity as a function of perylene concentration was drawn. Toluene, λₑₓ=650 nm, 100 mW cm⁻².

FIG. 7. Upconversion emission spectra of PdTPBP (10 μM) and perylene (250 μM) in argon and air. (solvent: toluene, λₑₓ=650 nm, 100 mW cm⁻²).

FIG. 8. Efficient triplet triplet energy transfer from PdTPBP to perylene. a) Phosphorescence quenching of PdTPBP (λₑₓ=635 nm) with the increasing perylene concentration in deaerated toluene. c (PdTPBP)=10 μM. b) The Stern-Volmer quenching curve of the phosphorescence of PdTPBP in the presence of perylene, in toluene. k_{q}=kₛᵥ/τ_{T}, k_{q} is bimolecular quenching constant, τ_{T} is the triplet excited lifetime of PdTPBP (τ_{T}=252 μs).

FIG. 9. Power-dependence TTA-upconversion of PdTPBP and perylene. a) TTA-upconversion intensity of PdTPBP (10 μM) and perylene (250 μM) dye pair in degassed toluene with different incident light power. b) Double-logarithmic plot of integrated emission intensity as a function of 650 nm excitation power density was drawn. Solid lines illustrate a slope of 2.27 (black, quadratic) and a slope of 0.87 (red, linear), Iₜₕ is 88.5 mW cm⁻².

FIG. 10. A picture of the TTA-UCNP that was prepared.

FIG. 11. The transmission electron microscope image of TTA-UCNP after negative staining with sodium phosphotungstate. Black line represents scale bar which is 100 nm.

FIG. 12. Normalized UV-vis absorption spectra of TTA-UCNP in the PBS buffer.

FIG. 13. The illustration of the process of TTA-UCNP in conjugation with GOX as the sensor to measure the glucose in aqueous solution.

FIG. 14. The phosphorescence intensity of PdTPBP under various conditions. a) The phosphorescence emission spectra of PdTPBP (10 μM) in toluene with air or argon. b) Phosphorescence emission spectra of PdTPBP NP under different conditions (glucose or GOX alone, in the absence of glucose and GOX, and in the presence of both glucose and GOX) in PBS buffer. c The kinetic process of PdTPBP NP in the presence of GOX (15 μg mL⁻¹) and glucose (5 mg mL⁻¹). The half response time is 25.1 s, λₑₓ=650 nm.

FIG. 15. Phosphorescence emission spectra of PdTPBP NP and the TTA-UCNP in the presence of glucose (5 mg mL⁻¹) and GOX (37.5 μg mL⁻¹). The phosphorescence emission spectra were recorded with a Hitachi F-7000 fluorescence spectrometer, λₑₓ=630 nm.

FIG. 16. The impact of GOX concentration on the upconversion of TTA-UCNP. a) The representative kinetic process of the TTA-UCNP in the different concentration of GOX (1.3, 3.9, 6.5, 13.0, 32.5 μg mL⁻¹) and glucose (5 mg mL⁻¹). b) The exponential relationship was found between the upconversion half response time and the concentration of GOX. λₑₓ=650 nm, 100 mW cm⁻², n=3 means that each experiment is repeated three times independently, the error bar represents the mean of the three times±standard deviation (SD).

FIG. 17. The TTA-upconversion emission spectra of the mixture of TTA-UCNP and GOX (35.2 μg mL⁻¹) in PBS buffer upon the addition of various analytes (0.7 mg mL⁻¹) including D-(+)-glucose, D-(−)-Fructose, D-Gluconic acid, 2-D-dexy-D-ribose, Glycerol, D-Mannitol, Glucuronolactone, Sucrose, Guanosine, Maltose, D-Lactose, and D-(+)-Raffinose. λₑₓ=650 nm, 100 mW cm⁻².

FIG. 18. TTA-upconversion spectra of TTA-UCNP in the presence of glucose (2 mg mL⁻¹) and GOX (10 μg mL⁻¹). λₑₓ=650 nm, 100 mW cm⁻², in cell culture medium including 5%, 10%, 15% and 20% FBS.

FIG. 19. The representative response times of the mixture of TTA-UCNP (1 mg mL⁻¹) and GOX (10 μg mL⁻¹) with different concentrations of glucose in the cell culture medium containing 20% FBS. λₑₓ=650 nm, 100 mW cm⁻².

FIG. 20. Ru NPs as the sensor for GOX sensing. a) UV-vis absorption spectrum of Ru NPs in deionized water. b) Phosphorescence emission spectra of Ru NPs in different conditions in deionized water. GOX (10 μg mL⁻¹), glucose (3.0 mg mL⁻¹). c) The response times of the mixture of Ru NPs and GOX (10 μg mL⁻¹) in the different concentrations of glucose. λₑₓ=460 nm. d) Phosphorescence emission spectra of Ru NPs in different conditions in the presence of cell culture medium. GOX (10 μg mL⁻¹), glucose (3.0 mg mL⁻¹). Ru NPs full name: Ru(bpy)₃ nanoparticles.

### DETAILED DESCRIPTION OF THE INVENTION

The invention is based in part on the discovery of novel and straightforward strategy to overcome the oxygen quenching problem to turn on the upconversion emissions of TTA-UCNP in the aqueous solution. The disclosed approach is based on the simple glucose oxidase (GOX) catalytic reaction, which directly exhaust and deplete the oxygen in the aqueous solution. Moreover, it was demonstrated that this method can be utilized in highly selective glucose sensors and to detect enzymes linked to glucose metabolism. It is believed to be the first demonstration of the long-thought after TTA-UCNP based solution as biological sensors. The disclosed invention should pave the way for using TTA-UCNP in a wide variety of photonic and biophotonic applications that are currently off limit due to the oxygen quenching problem.

Organic TTA-UCNPs based sensors are expected not only to inherit these general merits from inorganic lanthanide UCNPs (e.g., minimized autofluorescence background interference, reduced light scattering and photodamage), but also to have certain distinctive advantages over their inorganic counterparts in regard to biological sensing applications.

The emission of lanthanide UCNPs comes from a number of rare earth ions buried and shielded inside nanoparticles, which cannot respond and react directly to analytes in aqueous solution. In order to be used as biosensors, such inorganic UCNPs typically have to be conjugated with other fluorophore containing molecules (e.g., cyanine dye, rhodamine dye and ruthenium complexes) or by growing materials whose absorption overlaps with upconversion emissions. These inorganic UCNPs sensors rely on an inefficient luminescence resonance energy transfer (LRET) mechanism or on emission-reabsorption process. The readouts of such indirect sensors are often difficult to control due to variations regarding the amount and aggregation of conjugated dyes and the resultant materials. Since the ratios and intensities of these upconversion peaks and overall upconversion colors are heavily dependent on the power density of the excitation light and dopant amounts, the accuracy, sensitivity and robustness of such sensors can often be compromised by the slight variation in these experimental factors. (Wu, et al. 2015 Bioconjug. Chem. 26, 166-175; Punjabi, et al. 2014 ACS Nano. 8, 10621-10630; Liu, et al. 2013 J. Am. Chem. Soc. 135, 9869-9876; Wang, et al. 2017 J. Am. Chem. Soc. 139, 12354-12357; Liu, et al. 2014 J. Am. Chem. Soc. 136, 9701-9709; Deng, et al. 2011 J. Am. Chem. Soc. 133, 20168-20171; Peng, et al. 2017 Angew. Chem. Int. Ed. 56, 4165-4169; Tu, et al. 2014 Coord. Chem. Rev. 273, 13-29; Wen, et al. 2018 Nat. Commun. 9, 2415.)

In contrast, the emission of organic TTA-UCNP is based on the triplet excited states of photosensitizers and annihilators. Thus, similar to organic fluorescence molecules, there is typically one emission peak and no color mixing problem in organic TTA-UC. Unlike the inorganic UCNPs, organic TTA-UCNP can potentially be used as self-standing biosensors to detect analytes that can regulate and affect the triplet properties without any further nanoparticle modifications. In addition, compared to the weak absorption of lanthanides ions in inorganic UCNPs, TTA-UCNP utilizes intense long-wavelength absorbing organic photosensitizers, which lead to a much higher level of brightness and can be triggered with low power LED light.

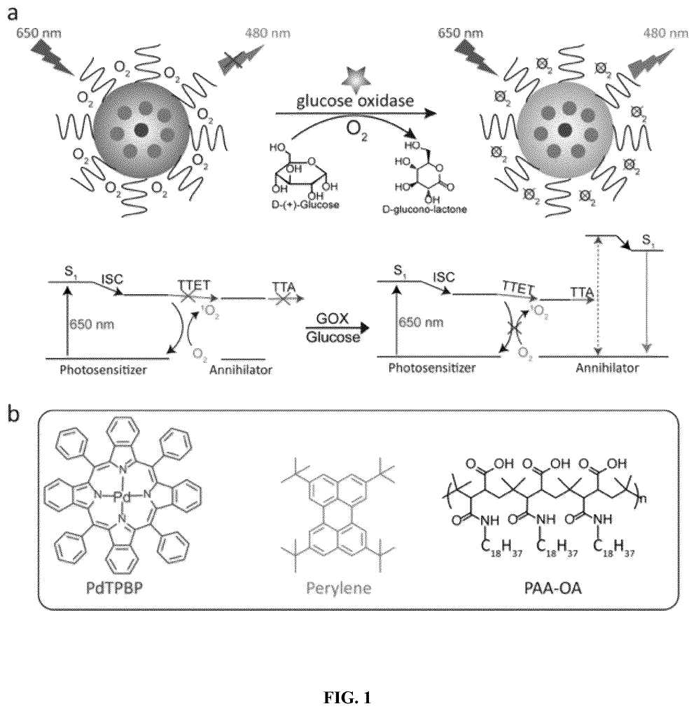

Glucose oxidase (GOX)-catalyzed glucose oxidation reactions can consume oxygen (FIG. 1a). GOX is biocompatible and has been widely used in medicine and biology. It is envisioned that such a reaction can be used to remove oxygen in aqueous solution to turn on and amplify the upconversion emissions of TTA-UCNP (FIG. 1a). (Shi, et al. 2018 Chem. Soc. Rev. 47, 4295-4313; Xiong, et al. 2015 Nanoscale, 7, 15584-15588; Qiao, et al. 2015 RSC Adv. 5, 69042-69046; Yoo, et al. 2010 Sensors 10, 4558-4576; Bankar, et al. 2009 Biotechnol. Adv. 27, 489-501; Raveendran, et al. 2018 Food Technol. Biotechnol. 56 16-30.)

The invention disclosed herein is a simple and straightforward GOX-catalytic method that can quickly deplete oxygen and thus amplify the upconversion intensity of sub-50 nm TTA-UCNP in aqueous solution. Additionally, self-standing “turn-on” upconversion biological sensors are developed based on such enzymatic TTA-UC enhancement strategy. It has been demonstrated that TTA-UCNP can detect glucose and measure the activity of enzymes related to glucose metabolism in a highly specific, sensitive and background-free manner. Thus, this disclosure not only provides a straightforward method for overcoming a key roadblock in using small-size TTA-UCNP for photonic and biophotonic applications in aqueous solutions, but also provides proof-of-concept for use of TTA-UCNP as background free self-standing biological sensors.

In one aspect, the invention generally relates to an upconversion nanoparticle comprising an organic photosensitizer and an organic annihilator pair encapsulated in a polymeric material, wherein upon excitation the organic photosensitizer-annihilator pair is capable of undergoing triplet-triplet annihilation upconversion to generate emission having a shorter wavelength than that of the excitation.

In certain embodiments, the organic photosensitizer is selected from the group consisting of: iridium complexes, metal-porphyrin complexes, metal-benzoporphyrin complexes, boron-dipyrromethene or derivatives thereof.

In certain embodiments, the organic photosensitizer is an iridium complex selected from:

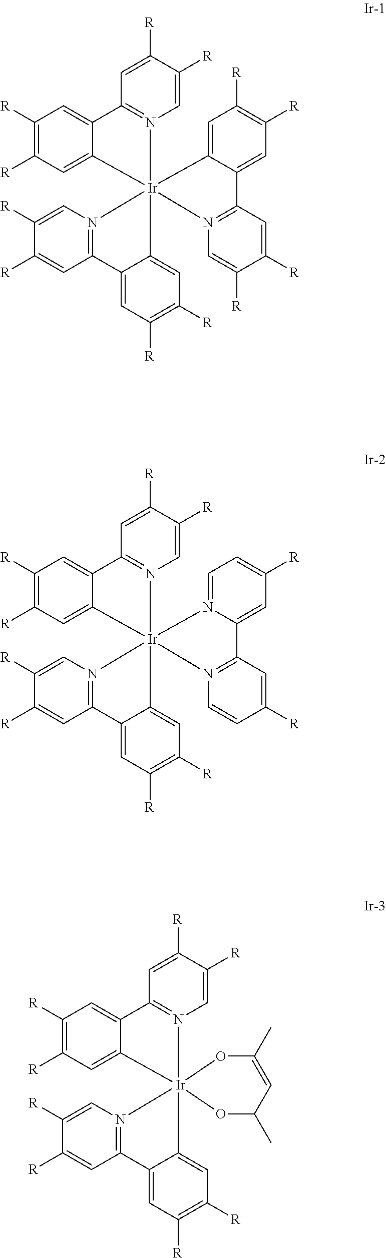

wherein

R is selected from the group consisting of: hydrogen, fluoro, trifluoromethyl, tert-butyl, alkyl, azide, polyethylene glycol (PEG), amine, carboxylic acid, sulfonate and hydroxyl.

Exemplary iridium complexes include:

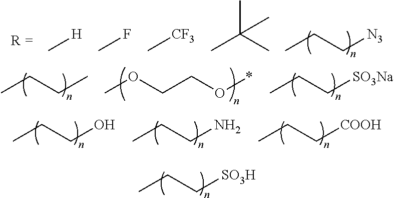

wherein n is an integer from 1 to 7.

In certain embodiments, the organic photosensitizer is a metal-porphyrin complex selected from:

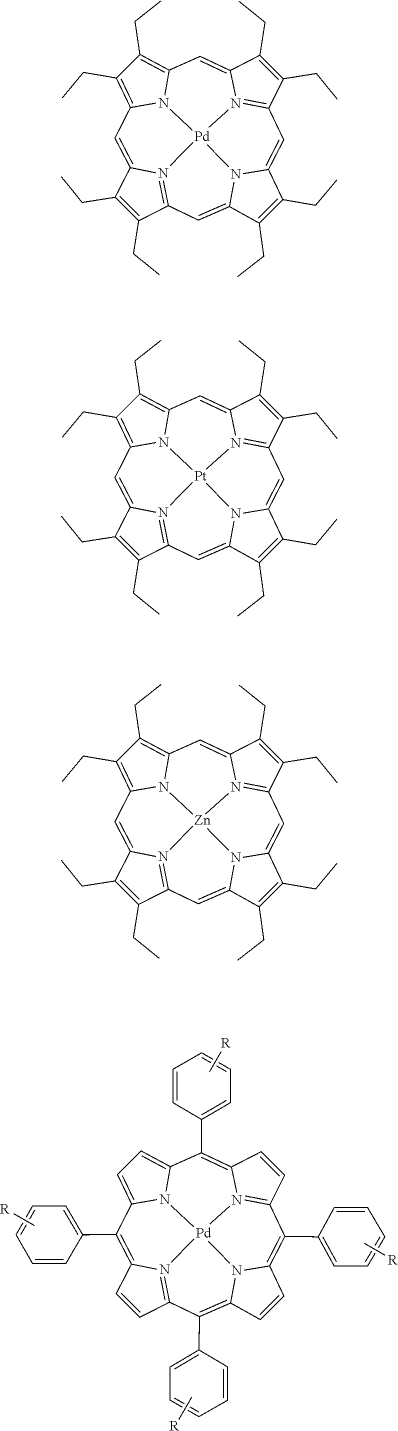

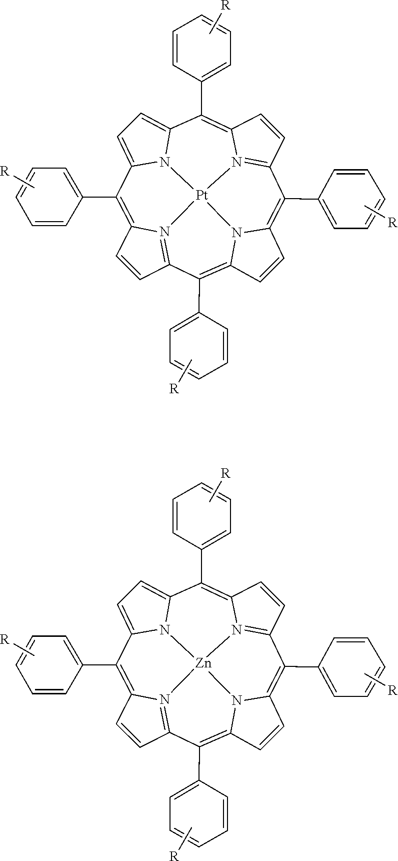

wherein
R is selected from the group consisting of: hydrogen, bromo, iodio, fluro, cyan, hydroxy, amino, alkynyl, alkyl, alkenyl, azide, PEG, amine, carboxylic acid, sulfonate and hydroxyl.

Exemplary metal-porphyrin complexes include:

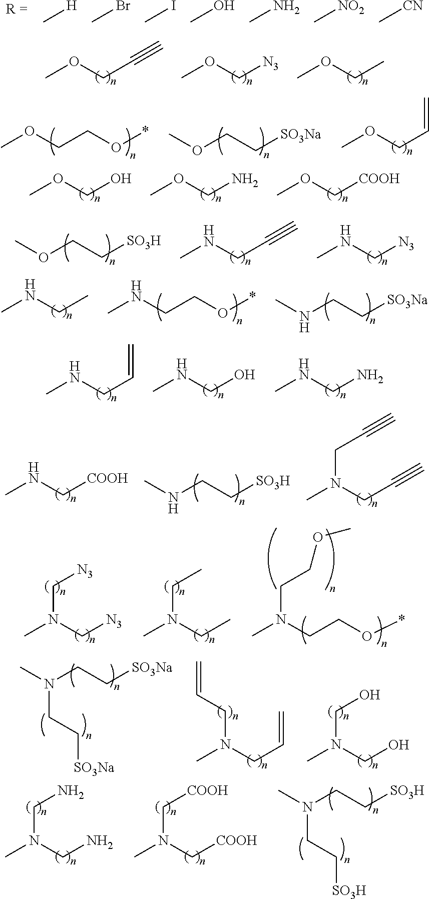

wherein n is an integer from 1 to 7.

In certain embodiments, the organic photosensitizer is a metal-benzoporphyrin complex selected from:

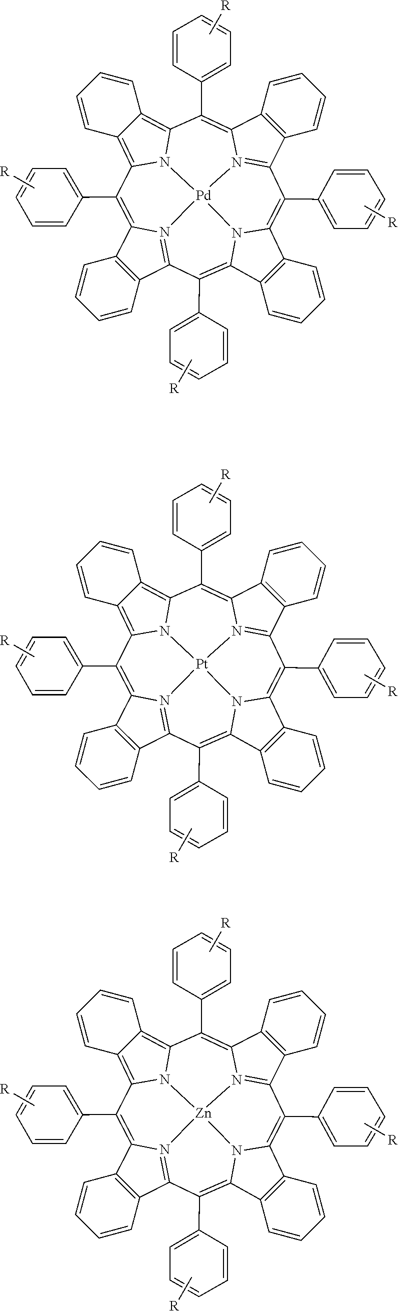

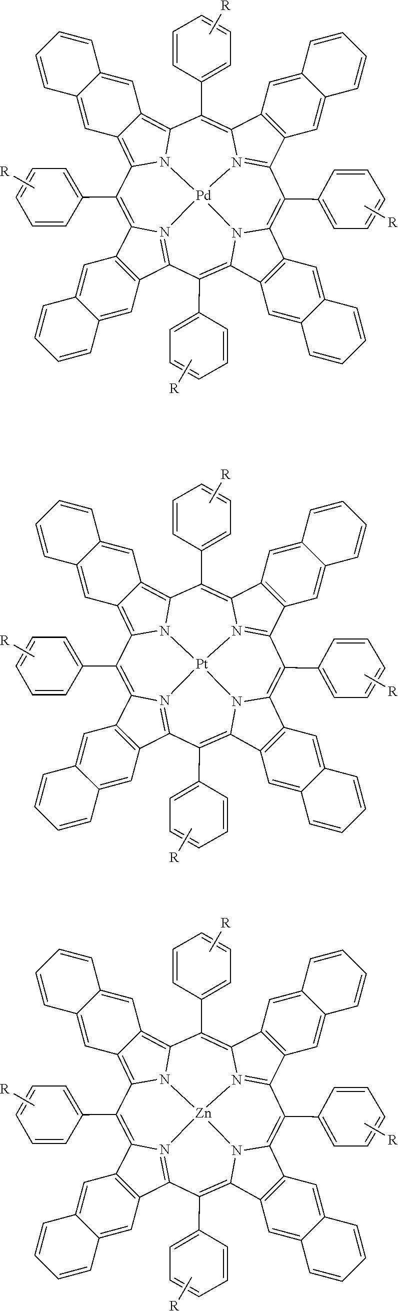

wherein

R is selected from the group consisting of: hydrogen, bromo, iodio, fluro, 1-naphthyl, 1-naphthalenyl, 1-naphthyloro, aldehyde, phenyl, styryl, phenylethynyl, alkynyl, vinyl, 2-naphthyl, 2-naphthalenyl, 2-naphthyl, 2-fluorenyl, 2-fluorenyl alkynyl, carbazolyl, carbazolyl alkynyl, alkynyl, hydroxy, amino, alkynyl, alkyl, alkenyl, azide, PEG, amine, carboxylic acid, sulfonate and hydroxyl.

Exemplary metal-benzoporphyrin complexes include:

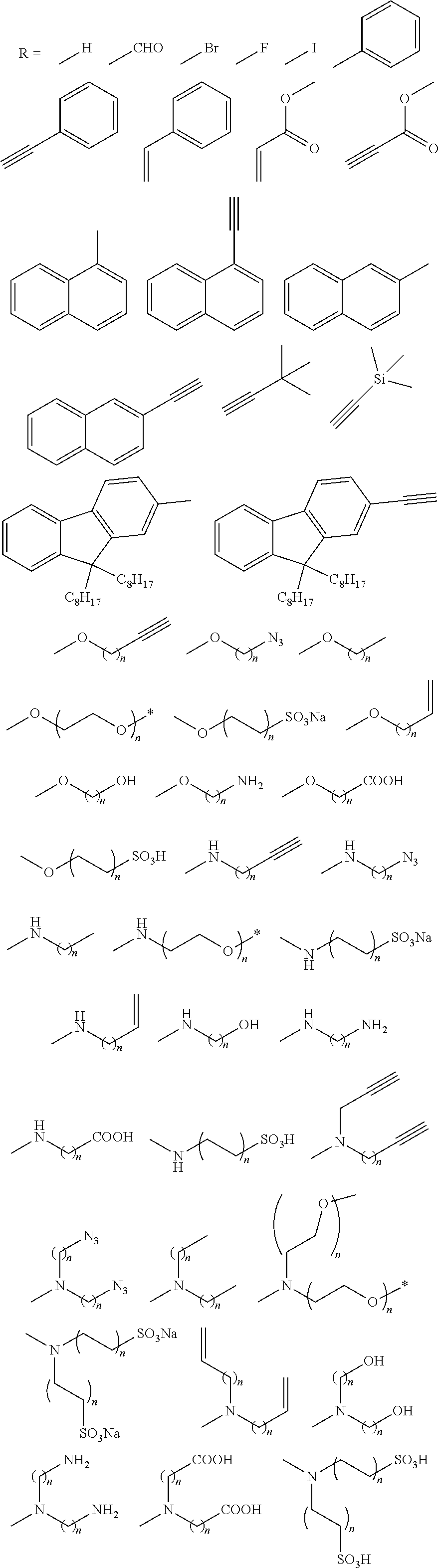

wherein n is 1, 2, 3, 4, 5, 6 or 7.

In certain embodiments, the organic photosensitizer is a boron-dipyrromethene derivative selected from:

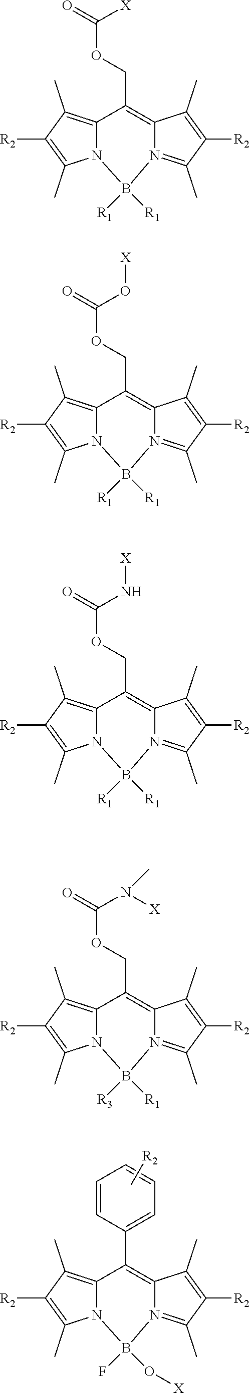

wherein
X is a targeted molecule;
R₁ is selected from the group consisting of: alkynyl, fluro and alkyl; and
R₂ is selected from the group consisting of: hydrogen, bromo, iodio, fluro, 1-naphthyl, 1-naphthalenyl, 1-naphthyloro, aldehyde, phenyl, styryl, phenylethynyl, alkynyl, vinyl, 2-naphthyl, 2-naphthalenyl, 2-naphthyl, 2-fluorenyl, 2-fluorenyl alkynyl, carbazolyl, carbazolyl alkynyl, alkynyl, hydroxy, amino, alkynyl, alkyl, alkenyl, azide, PEG, amine, carboxylic acid, sulfonate and hydroxyl.

Targeted molecules include biological active or functional small molecular drugs, proteins, peptides, amino acids, and nucleic acid molecules (e.g., DNAs).

Exemplary boron-dipyrromethene derivatives include:

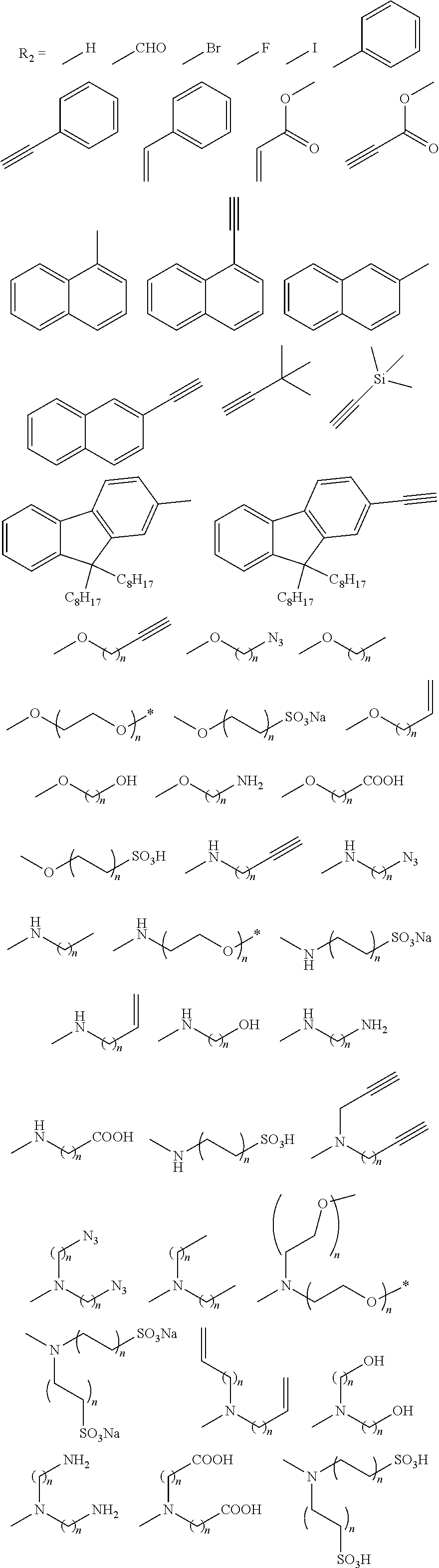

wherein n is 1, 2, 3, 4, 5, 6 or 7.

In certain embodiments, the organic photosensitizer is an iridium complex.

In certain embodiments, the organic photosensitizer is a metal-porphyrin complex.

In certain embodiments, the organic photosensitizer is a metal-benzoporphyrin complex.

In certain embodiments, the organic photosensitizer is a boron-dipyrromethene derivative.

In certain embodiments, the organic annihilator is selected from the group consisting of: coumarin or derivatives thereof, anthracene derivatives, perylene or derivatives thereof.

In certain embodiments, the organic annihilator is a coumarin derivative selected from:

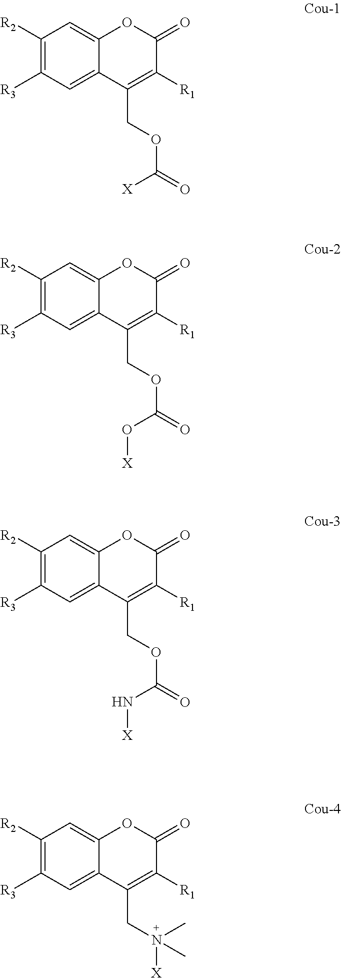

wherein

R₁ can be the following functional groups: hydrogen, bromo, iodio, and aldehyde, alkynyl and alkenyl;

R₂ can be the following functional groups: hydrogen, bromo, iodio, hydroxy, amino and nitro;

R₃ can be the following functional groups: alkynyl, alkyl, alkenyl, azide, PEG, amine, carboxylic acid, sulfonate and hydroxyl; and

R₄ can be the following functional groups: alkynyl, alkyl, alkenyl, azide, PEG, amine, carboxylic acid, sulfonate and hydroxyl.

Exemplary coumarin derivatives include:

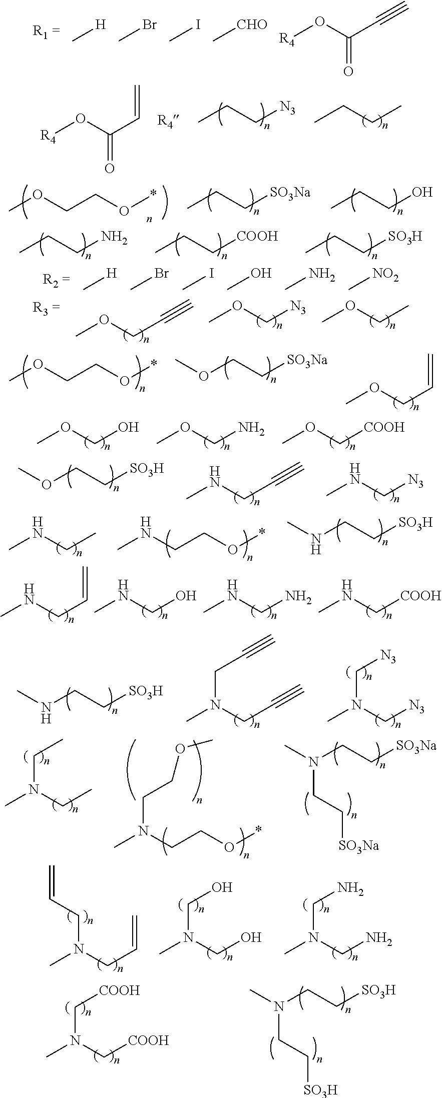

wherein n is 1, 2, 3, 4, 5, 6 or 7.

In certain embodiments, the organic annihilator is an anthracene derivative selected from:

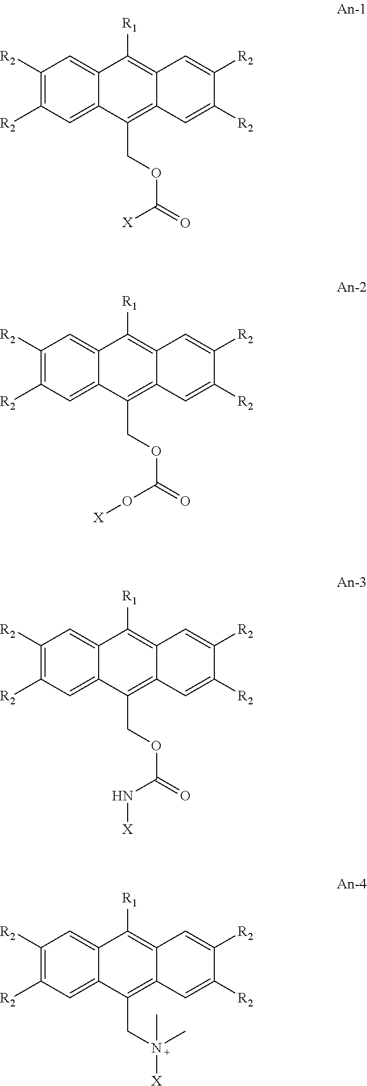

wherein
R₁ is selected from the group consisting of: hydrogen, bromo, iodio, fluro, 1-naphthyl, 1-naphthalenyl, 1-naphthyloro, aldehyde, phenyl, styryl, phenylethynyl, alkynyl, vinyl, 2-naphthyl, 2-naphthalenyl, 2-naphthyl, 2-fluorenyl, and 2-fluorenyl alkynyl; and
R₂ is selected from the group consisting of: hydrogen, fluro, hydroxy, amino, alkynyl, alkyl, alkenyl, azide, PEG, amine, carboxylic acid, sulfonate and hydroxyl.

Exemplary anthracene derivatives include:

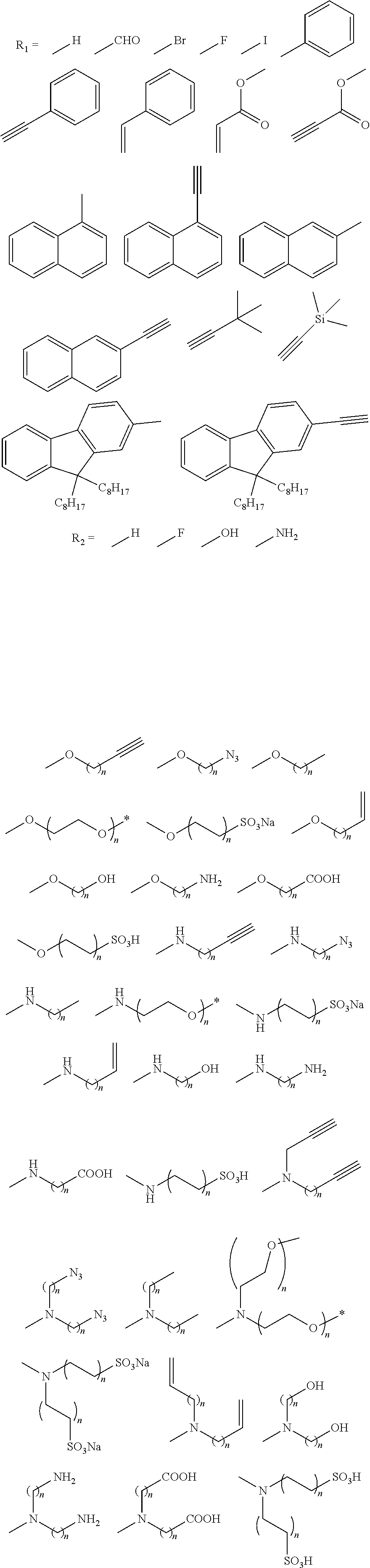

wherein n is 1, 2, 3, 4, 5, 6 or 7.

In certain embodiments, the organic annihilator is a perylene derivative selected from:

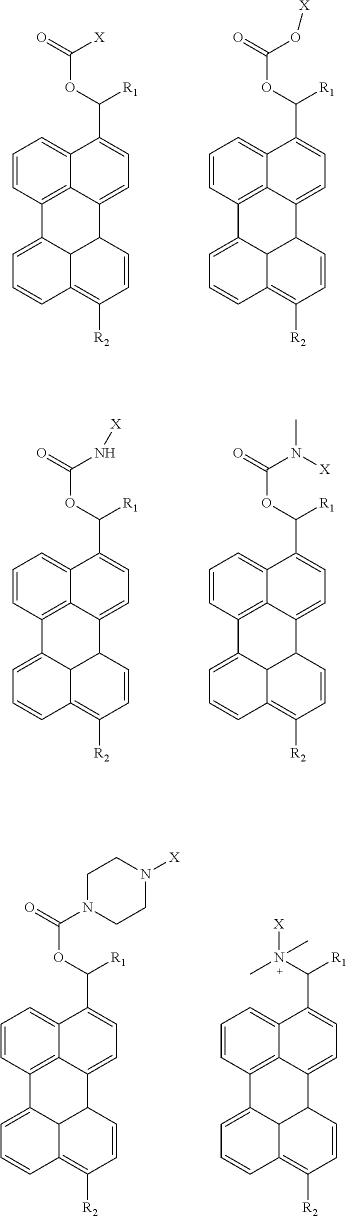

wherein
X is a targeted molecule;
R₁ is selected from the group consisting of: hydrogen, hydroxy, amino, alkynyl, alkyl, alkenyl, azide, PEG, amine, carboxylic acid and sulfonate; and
R₂ is selected from the group consisting of: hydrogen, bromo, iodio, fluro, 1-naphthyl, 1-naphthalenyl, 1-naphthyloro, aldehyde, phenyl, styryl, phenylethynyl, alkynyl, vinyl, 2-naphthyl, 2-naphthalenyl, 2-naphthyl, 2-fluorenyl, 2-fluorenyl alkynyl, carbazolyl and carbazolyl alkynyl.

Targeted molecules include biological active or functional small molecular drugs, proteins, peptides, amino acids, and nucleic acid molecules (e.g., DNAs).

Exemplary perylene derivatives include:

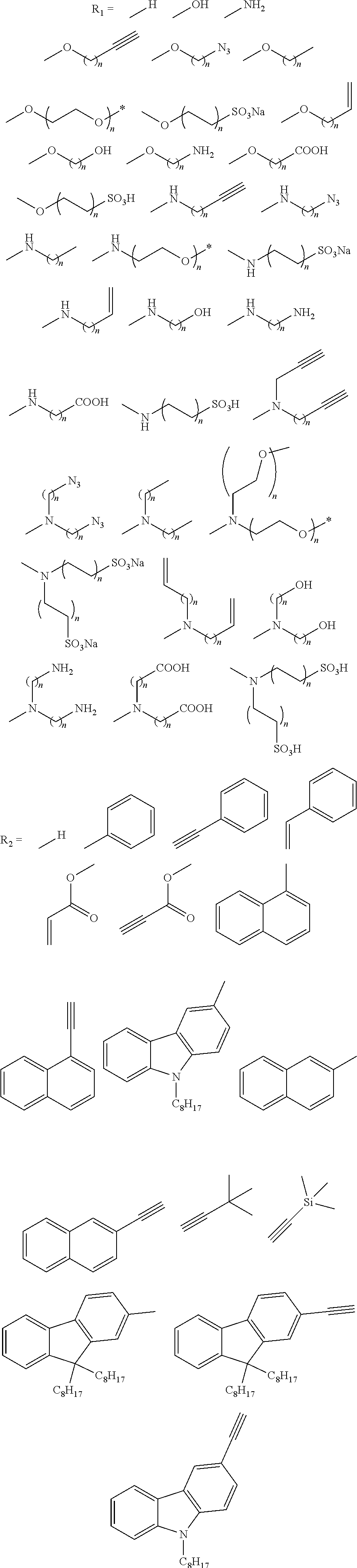

wherein n is 1, 2, 3, 4, 5, 6 or 7.

In certain embodiments, the organic annihilator is perylene or a derivative thereof

In certain embodiments, the organic photosensitizer is PdTPBP and the organic annihilator is perylene.

In certain embodiments, the polymeric material is an amphiphilic polymer selected from dodecane substituted PAA and octadecane substituted PAA.

In certain embodiments, the polymeric material is PAA-OA.

In certain embodiments, the upconversion nanoparticle has a particle size in the range of about 50 nm to about 200 nm (e.g., about 50 nm to about 100 nm, about 100 nm to about 150 nm, or about 150 nm to about 200 nm).

In certain embodiments, the upconversion nanoparticle has a particle size less than about 50 nm.

In certain embodiments, the upconversion nanoparticle is water dispersible.

In another aspect, the invention generally relates to an aqueous composition comprising a plurality of the upconversion nanoparticle disclosed herein.

In certain embodiments, the aqueous composition further comprises glucose (e.g., D-glucose).

In certain embodiments, the aqueous composition further comprises glucose oxidase.

In certain embodiments, the aqueous composition comprises glucose and glucose oxidase.

In yet another aspect, the invention generally relates to a sensor device that comprises upconversion nanoparticles or an aqueous composition disclosed herein.

In yet another aspect, the invention generally relates to a method for detecting glucose. The method comprises: providing an aqueous composition comprising upconversion nanoparticles disclosed herein and glucose oxidase; measuring a rate of triplet-triplet annihilation upconversion; adding a test sample to the aqueous composition; measuring the rate of triplet-triplet annihilation upconversion again; and analyzing the presence and/or concentration of glucose based on measurements with and without the test sample.

In yet another aspect, the invention generally relates to a method for measuring activity of an enzyme related to glucose metabolism. The method comprises: providing an aqueous composition comprising upconversion nanoparticles disclosed herein and glucose; measuring a rate of triplet-triplet annihilation upconversion; adding to the aqueous composition a test sample; measuring the rate of triplet-triplet annihilation upconversion again; and analyzing enzymatic activity based on measurements with and without the test sample.

In certain embodiments, measuring a rate of triplet-triplet annihilation upconversion comprises directing an excitation light beam corresponding to the singlet excitation state of the organic photosensitizer at the aqueous composition and measuring an emission therefrom at a wavelength longer than the excitation light beam.

In certain embodiments, analyzing the presence and/or concentration of glucose comprises measuring concertation of glucose half response time of triplet-triplet annihilation upconversion.

In certain embodiments, the organic photosensitizer is PdTPBP, the organic annihilator is perylene, and the polymeric material is PAA-OA.

In certain embodiments, the excitation light beam has a wavelength at about 650 nm and the emission has a wavelength at about 480 nm.

In certain embodiments, the enzyme related to glucose metabolism is selected from invertase (β-fructofuranosidase), glucose dehydrogenase and gluconate kinase.

In certain embodiments, the enzyme related to glucose metabolism is invertase (β-fructofuranosidase).

The following examples are meant to be illustrative of the practice of the invention and not limiting in any way.

### EXAMPLES

### Synthesis and Characterization of Water-Dispersible TTA-UCNP

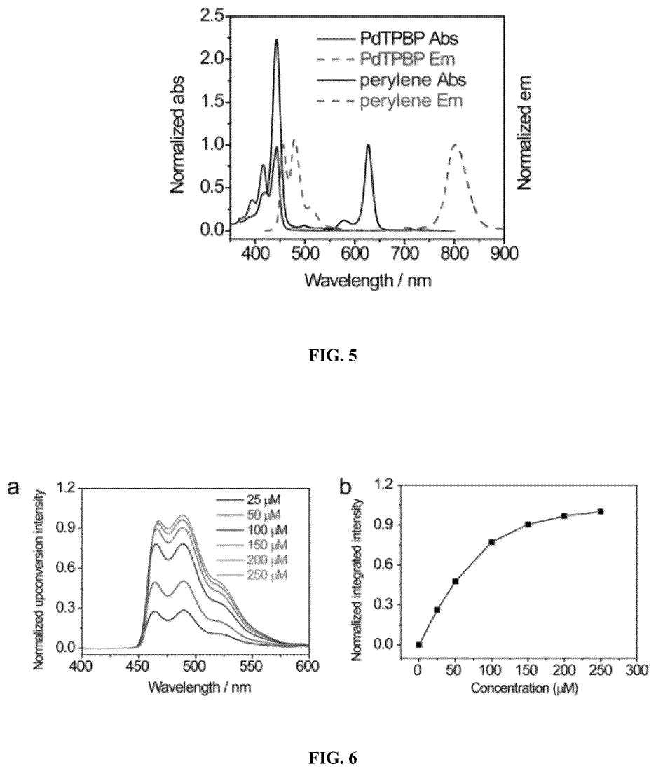

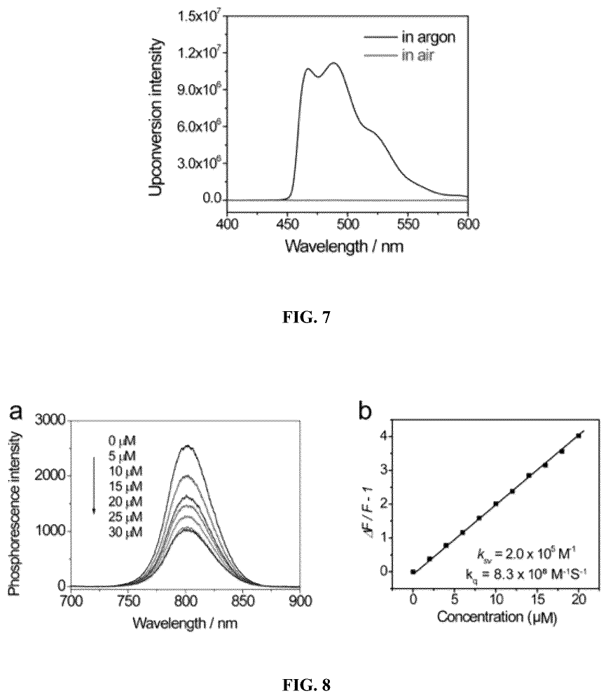

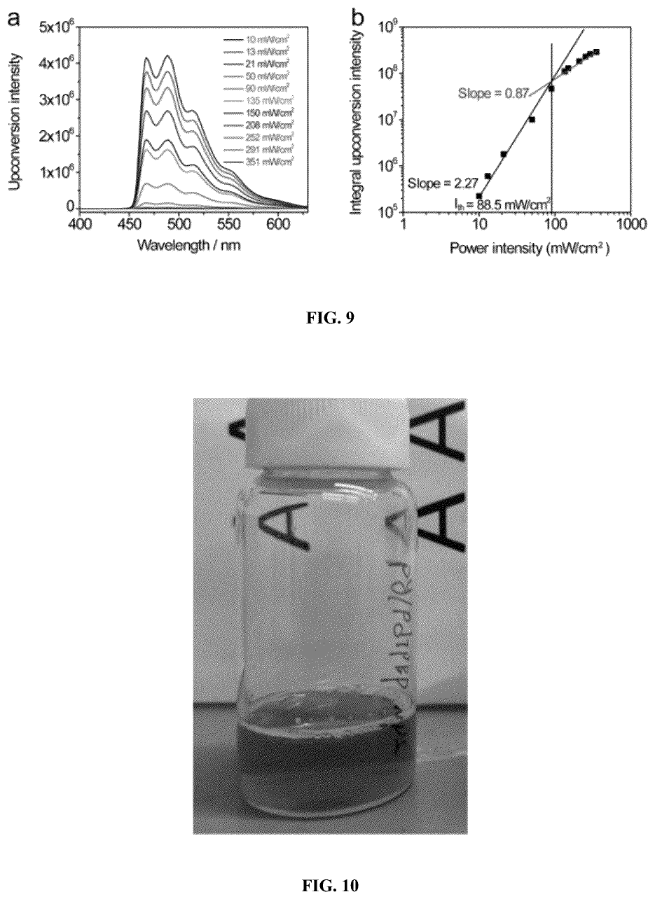

As a proof-of-principle, palladium (II) meso-tetraphenyl-tetrabenzoporphyrin (PdTPBP) and perylene were used as the sensitizer and annihilator, as they are one of the most investigated and effective long wavelength activating TTA-UC dye pairs used in organic solvent. As the annihilator, perylene has (FIG. 5) high fluorescence quantum yield (Φ₇₁ =98%) and robust photostability. PdTPBP is a potent far-red light absorbing photosensitizer and its maximum absorption peak is located at 628 nm (molar extinction coefficient, ε=1.1×10⁵ M⁻¹ cm⁻¹) (FIG. 5). Moreover, the triplet excited state energy level (T₁) of perylene is 1.52 eV, which is energetically compatible with that of PdTPBP (1.55 eV). In the present study, the optimal concentrations of PdTPBP and perylene is determined to be 10 μM (PdTPBP) and 250 μM (perylene) (FIG. 6). Intense far-red to blue TTA upconversion emission can be observed with upconversion quantum yield up to 7.9% in deaerated toluene (FIG. 7). The triplet-triplet energy transfer (TTET) rate between PdTPBP and perylene was found to be high (kₛᵥ=2.0×10⁵ M⁻¹), as determined via Stern-Volmer quenching curves (FIG. 8). The power-dependent TTA-UC spectra were further tested and the TTA-UC threshold intensity is observed to be 88.5mW cm⁻² (FIG. 9).

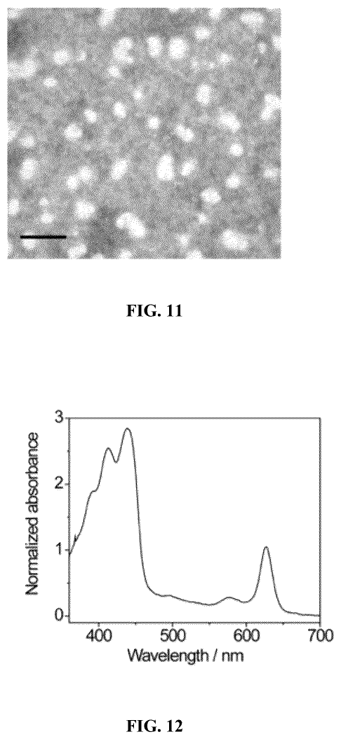

To obtain a water-dispersible TTA-UCNP, this TTA dye pair (PdTPBP/perylene) was encapsulated (FIG. 10) in an amphiphilic polymer-polyacrylic acid substituted octadecylamine (PAA-OA). The size of the TTA-UCNP was estimated by transmission electron microscopy (TEM) to be 40.2±3.2 nm (FIG. 11). The hydrodynamic diameter of the TTA-UCNP was determined to be 57.3±3.9 nm using dynamic light scattering. The UV-vis absorption spectra of the TTA-UCNP was also measured. As shown in FIG. 12, the TTA-UCNP presents the characteristic absorption peak from perylene at 440 nm and the characteristic absorption peak of PdTPBP at 628 nm (FIG. 5), suggesting that the TTA-UCNP encapsulated both the photosensitizer and annihilator. The entrapment efficiency of the TTA-UCNP is determined to be 92% (PdTPBP) and 75% (perylene).

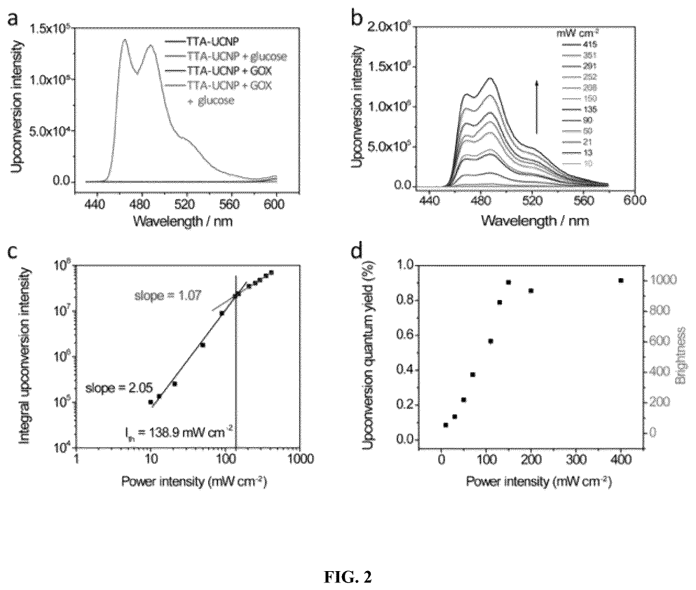

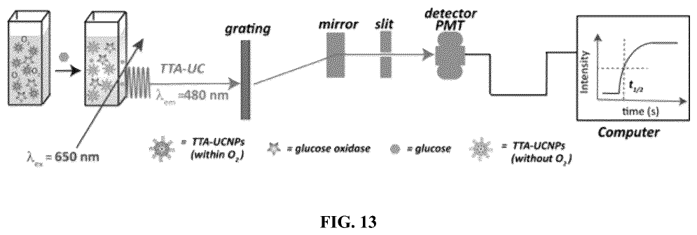

Next, the photophysical properties of this TTA-UCNP in phosphate-buffered saline (PBS) were investigated. The schematic diagram of instrument setup was presented on FIG. 13. As expected, in the absence of GOX or glucose, the upconversion emissions of nanoparticles in PBS buffer were not detectable. In contrast, in the presence of both GOX and glucose, intense blue upconversion emission was observed at 480 nm (FIG. 2a). Using well-established literature reported method, the intermolecular triplet energy transfer quantum yield from PdTPBP to perylene in the TTA-UCNP is determined to be 49.3%. Moreover, the upconversion emission for the TTA-UCNP in PBS is power-dependent with a threshold intensity (Iₜₕ) of 138.9 mW cm⁻². Below 138.9 mW cm⁻², the upconversion emission intensity (I_{UC}) and the excitation intensity (Iₑₓ) are in a quadratic relationship. When the Iₑₓ exceeds the threshold intensity, I_{UC} and Iₑₓ are linear (FIG. 2c). The upconversion quantum yield (Φ_{UC}) and respective brightness (η=ε×Φ_{UC}) of the TTA-UCNP in the presence of GOX and glucose were also measured and calculated. Below Iₜₕ, as the value of Iₑₓ increases, both the upconversion performance and the brightness increase. Beyond Iₜₕ, both two values reach plateau (Φ_{UC}=0.9% and η=1001) (FIG. 2d). Notably, under the low-power excitation light, the TTA-UC is determined by intermolecular collision between triplet excited state of annihilators (³[An]*), which is a bimolecular process. Thus, the power of the excitation light and the upconversion intensity have a quadratic relationship. In contrast, in high power excitation light, the intensity of TTA-UC typically depends on the radiation transition of the annihilator from the singlet excited state (¹[An]*) to the ground state, which is a single molecule process. Therefore, when the intensity of the excitation light is higher than Iₜₕ, the power of the excitation light and the upconversion intensity have a linear relationship and the TTA-UC quantum efficiency plateaus.

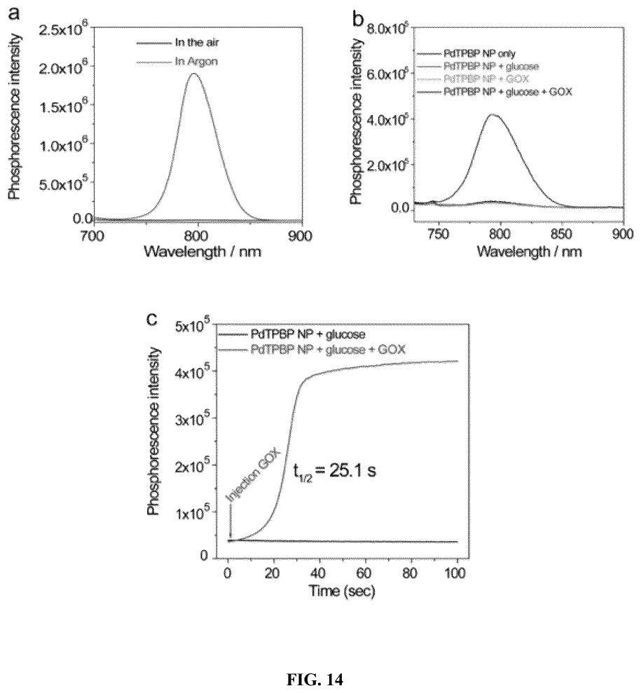

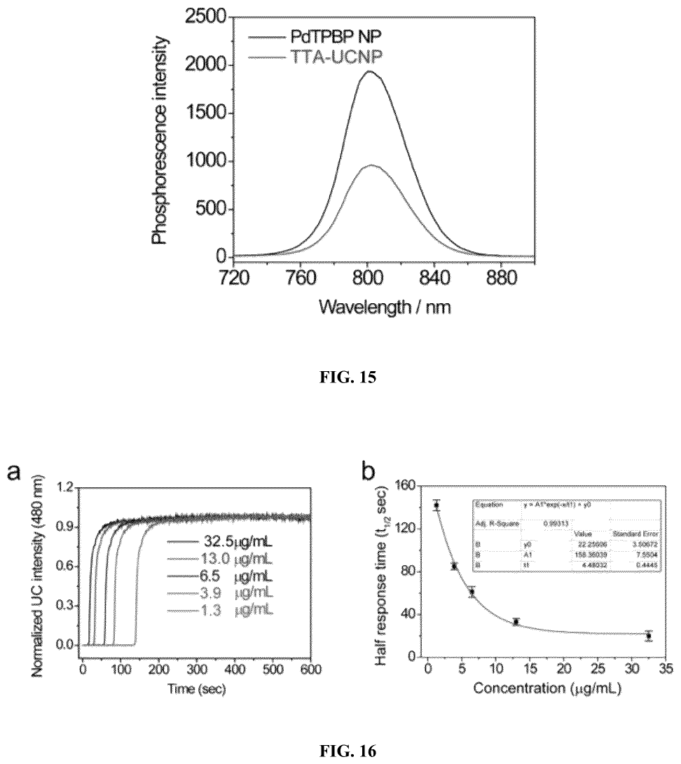

Since the phosphorescence of PdTPBP is sensitive to oxygen (FIG. 14a), a nanoparticle was also prepared with only PdTPBP that are coated by PAA-OA (PdTPBP NP) as a reference to further explore the mechanism of oxygen-clearance. Only weak phosphorescence was observed at 796 nm for PdTPBP NP in the absence of glucose and GOX (FIG. 14b). This suggests that the PAA-OA polymer cannot solve the oxygen quenching problem per se. Additionally, in the presence of only GOX or only glucose, the phosphorescence of the PdTPBP NP is also not enhanced, indicating that only GOX or glucose cannot clear oxygen in aqueous solution. In contrast, the phosphorescence of PdTPBP was effectively turned on with both GOX and glucose, indicating that oxygen consumption by GOX-catalyzed glucose oxidation took place (FIG. 14b). Moreover, in the presence of GOX and glucose, it was observed that the characteristic phosphorescence intensity of PdTPBP in TTA-UCNP is significantly lower than that in PdTPBP NP (FIG. 15). This also supports the efficient triplet-triplet energy transfer from PdTPBP to perylene inside TTA-UCNP.

Moreover, the influence of GOX concentration on the TTA-UC was studied by detecting the kinetic rates of TTA-UC emission intensity for these TTA-UCNP under 650 nm illumination. This was done with different concentrations of GOX and the fixed concentration of glucose (5 mg mL⁻¹). A higher concentration of GOX was found to lead to a decreased upconversion half response time (t_{½}). For example, t_{½} was shortened from 142 seconds to 20 seconds when the GOX concentration increased from 1.3 μg mL⁻¹ to 32.5 μg mL⁻¹ (FIG. 16a). The fitting curve of tin and the concentration of GOX displayed exponential relation (FIG. 16b), revealing that the enhancement of TTA-UC emission depends on the rate of oxygen consumption via the GOX catalyzed glucose oxidation reaction. The abovementioned results not only further verified that oxygen in the solution was consumed by the GOX-catalyzed glucose oxidation reaction, leading to upconversion emission “turn on”, it also demonstrated that the upconversion response time was actually tunable by controlling the concentration of GOX in the TTA-UCNP aqueous solution.

### TTA-UCNP for Glucose Sensing

Next, the feasibility of using this TTA-UC enhancement strategy to sense glucose in aqueous solution was explored. D-glucose has played key roles in a wide variety of biological processes in living organisms. An abnormal level of glucose in body fluid has been linked to several life-threatening diseases, such as diabetes, nephritis, as well as liver damage. Therefore, glucose sensing has considerable importance in the biomedical field. (Zimmet, et al. 2001 Nature 414, 782-787; Vallon, et al. 2020 Nat. Rev. Nephrol. 16, 317-336; Targher, et al. 2018 Nat. Rev. Endocrino. l. 14, 99-114; Shokrekhodaei, et al. 2020 Sensors 20, 1251.)

Prior to the present disclosure, the fluorescence glucose sensors were developed according to the capability of organic boronic acids to act as molecular receptors for saccharides, especially glucose. Yet, the affinity of boronic acid towards glucose are not optimal and its glucose selectivity is not specific. Meanwhile, in such a strategy, the conjugated reporter chromophores, such as anthracene, phenanthrene and naphthalene, are typically involved in short wavelength light excitation and emission (ultraviolet or blue light), the latter of which suffers from autofluorescence background from the biological fluids. Additionally, fluorescent probes or nanoparticles (gold or silver) were also used to indirectly estimate the amount of glucose by responding to H₂O₂ or protons (the products of the GOX catalytic glucose oxidation reactions). However, almost all these reported optical glucose sensors are based on changes in “downconverting” fluorescence or absorbance. Therefore, background interference remains and greatly reduces the specificity and sensitivity of glucose. Since upconversion materials have opposite optical profiles compared to conventional fluorescence chromophores, it was envisioned that unique GOX mediate TTA-UC enhancement can be used as a background-free glucose sensor. (Sun, et al. 2015 Chem. Rev. 115, 8001-8037; Steiner, et al. 2011 Chem. Soc. Rev. 40, 4805-4839; Guan, et al. 2013 Chem. Soc. Rev. 42, 8106-8121; Sedlmeier, et al. 2015 Chem. Soc. Rev. 44, 1526-1560; Chen, et al. 2014 Chem. Rev. 114, 5161-5214; Chen, et al. 2016 Chem. Rev. 116, 2826-2885.)

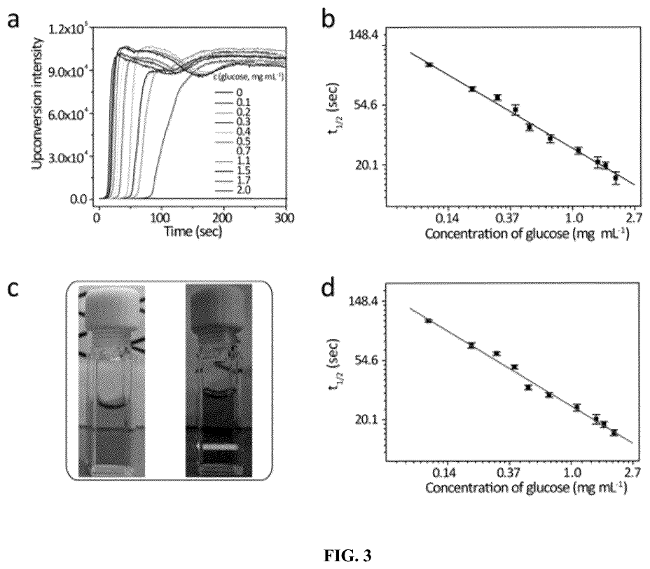

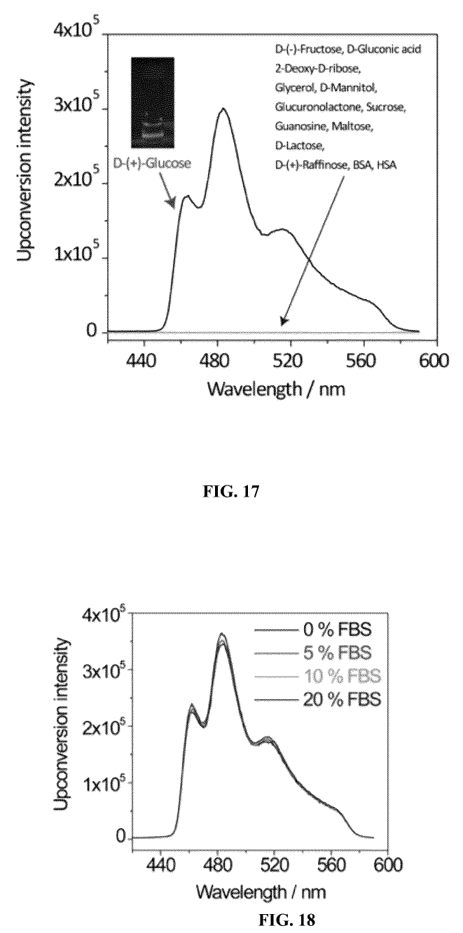

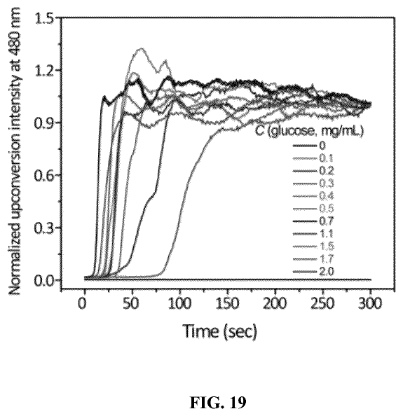

As has been shown in above-mentioned experimental results (FIG. 16), GOX concentrations dictate kinetic rates of the recovery of TTA-UC emission in the presence of glucose. Thus, for the purpose of glucose sensors, since the lower or higher end concentrations of GOX can lead to overly slow or fast readouts and subsequent potential experimental inconvenience and errors, GOX of 10 μg mL⁻¹ was chosen to demonstrate the glucose sensing ability of the TTA-UCNP. Glucose at different concentrations was added to a solution of GOX (10 μg mL⁻¹) and TTA-UCNP (1 mg mL⁻¹). It was observed that the response times for the TTA-UCNP to approach its maximal upconversion intensity are dependent on the glucose concentration. More specifically, the half response time is found to be linearly decreased with the increased concentration of glucose in a log scale manner (FIG. 3). The limit of detection for such glucose sensing are deduced to be 0.06 mg mL⁻¹. In addition, by linear extrapolating the fitted straight line (FIG. 3b), this concentration of detection is observed to be up to 3.0 mg mL⁻¹. This concentration range (0.06-3 mg mL⁻¹) is wider than the requirement for clinical fasting blood glucose (0.7-1.1 mg mL⁻¹) testing. Furthermore, the TTA-UCNP glucose sensors are found to be highly selective for glucose (FIG. 17). In this regard, a wide variety of other prevalent biological saccharides and polyhydric alcohols were also tested, such as fructose, 2-deoxy-d-ribose, sucrose, maltose, D-lactose, and D-(+)-raffinose, sodium D-gluconic acid, glycerol, D-mannitol, and glucuronolactone (FIG. 17). As a result, insignificant upconversion emissions were observed with these compounds. The upconversion emission response was only observed with glucose. Moreover, in the presence of 5%, 10% and 20% FBS respectively, there is no obvious change for TTA-upconversion intensity, suggesting that serum proteins have not significantly affect TTA-UCNP (FIG. 18). In addition, in order to explore whether the disclosed method can be interfered by the autofluorescence background, the tests were conducted in the colored cell culture containing 20% FBS, phenol red, and GOX (10 μg mL⁻¹). With the addition of glucose (2 mg mL⁻¹), obvious TTA-upconversion was observed (FIG. 3c, right). Then, under this condition, the half response time of TTA-upconversion at different concentrations of glucose (FIG. 19) was quantitatively measured. The concentration of glucose and half response time was fitted in In-In coordination (FIG. 3d) and then compared it to FIG. 3b (in deionized water, slope=−0.627), observing an insignificant change in slope (−0.633), which suggested that the TTA-UCNP can still accurately measure the glucose concentration with autofluorescence background. Compared to the typical examples of pre-existing glucose quantitative analysis methods (Stokes-emission-based techniques) (Table 1), the presently disclosed method, not only has a low detection line, it also has a wider detection range.

TABLE 1
Typical examples to detect glucose in the literatures.

| Sensor | Excitation/Emission | AR: analytical range | |
|---|---|---|---|
| GOx and probe Ru(phen) ^{a} | 488/610 nm | 0.7-10 | mM |
| GOx and Ru(phen) ^{b} | 468/570 nm | 0.5-15 | mM |
| EuTC and Hypan ^{c} | 400/616 nm | 0.1-2 | mM |
| Anthracene based bis-PhBA ^{d} | 370/423 nm | 0.3-1 | mM |
| TTA-UCNP (this work) | 650/480 nm | 0.33-17 | mM |

^{a} Rosenzweig, et al. 1996 Anal. Chem. 68, 1408-1413.
^{b} Bukowski, et al. 2007 Electron. Lett. 43, 202-204.
^{c} Schäferling, et al. 2004 J. Fluoresc. 5, 561-568.
^{d} James, et al. 1995 J. Am. Chem. Soc. 117, 8982-8987.

Notably in previous studies, it was necessary to embed the chromophores, such as Ru, Pd, Pt complexes and GOX, in a polymer or hydrogel in order to make the device measure the glucose content. (Steiner, et al. 2011 Chem. Soc. Rev. 40, 4805-4839.) In contrast, TTA-UCNP can be directly used to measure the glucose in aqueous solution without the requirement of a complicated device preparation process. For the purpose of direct comparison, as an example, the system disclosed herein was compared head-to-head with a well-established Ru(bpy)₃ in a nanoparticle formation to measure the glucose in the aqueous solution. (Moreno-Bondi, et al. 1990 Anal. Cham. 62, 2377-2380; Li, et al. 1995 Anal. Chem. 67, 3746-3752.)

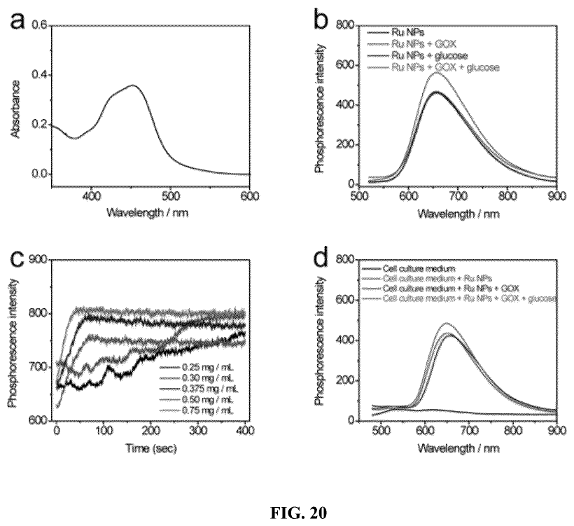

In this controlled study, Ru NPs were constructed like the protocol with TTA-UCNP. As shown in FIG. 20, in the presence of glucose and GOX, the phosphorescence intensity of Ru(bpy)₃ increases by 20% in DI water. However, such Ru NPs is not sensitive enough to quantitatively analyze the concentration of glucose below 1.6 mM, which can be well detected following the method disclosed herein. Moreover, in the presence of interference by the cell culture medium, the performance of such Ru(bpy)₃ is even worse. Only a 10% phosphorescence increase was observed in the presence of glucose and GOX and the detection was significantly impaired by the background.

### TTA-UCNP for the Measurement of Invertase Activity

Next, TTA-UCNP was studied in conjugation with GOX as sensors in order to measure the activity of enzymes related to glucose metabolism. As a proof-of-principle, the activity of the invertase (β-fructofuranosidase) was studied using the TTA-UCNP sensor. The invertase is an important enzyme that catalyzes hydrolysis of sucrose to produce D-glucose and D-fructose. These enzymes broadly exist in plants, microorganisms, and humans. In particular, the activity of such an enzyme must be routinely measured in the many areas of biology, and bioengineering. To date, invertase activity is determined by a couple of enzyme assays in which invertase cleaves sucrose to glucose and fructose, resulting in a colorimetric product, proportional to the invertase activity present. However, these existing methods are based on additional colored indicators, such as oxiRed probe (absorbance at 570 nm) to determine invertase activity. Moreover, there are typically biological pigments in the samples, such as chlorophyll, carotenes and phytochromes, which have significant interference when measuring the activity of invertase. (Roitsch, et al. 2004 Trends Plant Sci. 9, 606-613; Lincoln, et al. 2017 J. Basic. Microbiol. 57, 803-813; Kulshrestha, et al. 2013 J. Pharm. Res. 7, 792-797; Dambrouck, et al. 2005 J. Agric. Food. Chem. 53, 8782-8789; Romero-Gómez, et al. 2000 Biotechnol. Lett. 22, 1255-1258; Aguiar, et al. 2014 Mol Biotechnol. 56, 524-534; Shivalingamurthy, et al. 2018 Front Plant Sci. 9, 598; The commercialized products for invertase activity assay kit (sensitive): https://www.abcam.com/invertase-activity-assay-kit-colorimetric-ab197005.html.)

Contrary to these existing methods, the unique TTA-UC properties in the disclosed method is expected to overcome the problematic background interference and to minimize cross-talking with auto-fluorescence from the samples.

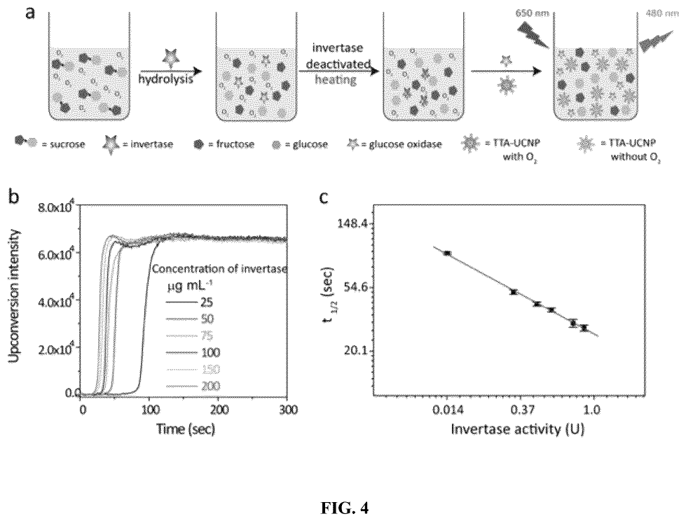

As shown in FIG. 4a, the sucrose and invertase were incubated at room temperature for 5 min. Then, the invertase was deactivated in boiling water for 10 min. When the solution cooled to room temperature, it was added with the TTA-UCNP (1 mg mL⁻¹) and GOX (10 μg mL⁻¹) (FIG. 4a). As shown in FIG. 4b, it was observed that the enzymatic reaction of the invertase can effectively turn on the original latent upconversion emissions of the TTA-UCNP. The higher concentration of invertase is found to lead to a quicker TTA upconversion response. Based on the results that can be seen in FIG. 3b, the concentrations of intermediate product, glucose, was calculated and then converted to the invertase activity. As shown in FIG. 4c, the invertase activity has a good linear relationship with half response time of TTA upconversion in In-In coordinates. The minimum detection limit is calculated to be 0.01 unit, which outperformed that of the commercial products (0.02 unit). (The commercialized products for invertase activity assay kit (sensitive): https://www.abcam.com/invertase-activity-assay-kit-colorimetric-ab197005.html.) These results show that the TTA-UCNP biosensors can also be extended to measure enzymatic activity that is related to glucose metabolism.

### Chemicals

Perylene, octadecylamine, poly (isobutylene-alt-maleic anhydride) M_{w}=6000, tetrahydrofuran (THF), N, N-dimethylformamide (DMF), glucose, glucose oxidase and invertase were purchased from Sigma-Aldrich (St. Louis, MO, U.S.). Meso-tetraphenyl-tetrabenzoporphine palladium complex (PdTPBP) was purchased from Fisher Scientific. Ultrapure water was prepared by using a Millipore Simplicity System (Millipore, Bedford, U.S.). All of the above-mentioned chemicals were used as received without further purification.

### Characterization

UV-vis spectra were recorded via an Agilent Cary-5 spectrophotometer. Steady-state fluorescence spectra were measured with a Hitachi F-7000 fluorescence spectrometer. The morphology of the TTA-UC nanoparticle was characterized by using a JEOL JEM-200CX transmission electron microscope (TEM) operated at 80 kv. The sample for TEM measurement was prepared by dropping the solution onto a carbon-coated copper grid after negative staining with 10.0% (w/v) sodium phosphotungstic acid. The particle size and size distribution of the TTA-UCNP in aqueous solution was measured by using dynamic light scattering (DLS) via a Malvern Zetasizer Nano ZS. The original data is re-drawn and analyzed on Origin Pro 8.

### PAA-OA Synthesis

Poly (isobutylene-alt-maleic anhydride) (M_{w} 6000) (1.85 g) and octadecylamine (1.6 g) were dissolved in dry THF (100 mL). They were then left to react for 24 h at 70° C. The solvent was evaporated under reduced pressure to attain the PAA-OA product.

### Synthesis of TTA-UCNP

The TTA-UCNP was prepared via self-assembly of the sensitizer (PdTPBP), the annihilator (perylene) with PAA-OA. Briefly, PdTPBP (50 μM), perylene (1 mM) and PAA-OA (75 mg) were dissolved in 5 mL THF, and followed by the addition of 5 mL 50 mM sodium borate buffer (with a pH of 9.16). The mixture solution was stirred in 40° C. for 2 h and the volume of the solution is reduced due to the evaporation of the THF. The reaction mixture was then cooled to room temperature and dialyzed in deionized water for 24 h. After that the nanoparticles were concentrated to 5 mL via an ultracentrifuge tube (cut off M_{w}=3000). The TTA-UCNP solution was then stored at 4° C. The PdTPBP nanoparticles (PdTPBP NP) was prepared by a similar method. PdTPBP (50 μM) and PAA-OA (75 mg) were used for preparing PdTPBP NP.

### TTA Upconversion Measurement in Toluene

A continuous diode-pumped solid-state laser (650 nm) was used as the excitation source for the upconversion measurement. For the upconversion measurements, the mixed solution of PdTPBP and perylene was degassed with Argon for 15 min. The solution was then excited with the laser, and the corrected upconversion spectrum was recorded with a HORIBA spectrofluorometer.

### Measurement and Calculation of Upconversion Quantum Yield (Φ_{UC})

The Φ_{UC} was calculated by an established method. (Singh-Rachford, et al. 2010 Coord. Chem. Rev. 254, 2560-2573; Huang, et al. 2017 Angew. Chem. Int. Ed. 56, 14400-14404.) The methylene blue (MB) in methanol with fluorescence quantum yield (Φ₇₁ =3%) was used as the reference. The upconversion quantum yield was calculated with Equation (1), where Φ_{UC}, A_{std}, I_{std}, and η_{std} represent the upconversion quantum yield, the absorbance of the reference, the integrated photoluminescence intensity of the reference, and the refractive index of the solvents:

$${Φ}_{UC} = 2 \times {Φ}_{f} \times ( \frac{{A}_{std}}{{A}_{unk}} ) \times ( \frac{{I}_{unk}}{{I}_{std}} ) \times {( \frac{{η}_{unk}}{{η}_{std}} )}^{2} \qquad ( 1 )$$

where Φ_{UC}, A_{std}, I_{std}, and ƒ_{std} represent the upconversion quantum yield, the absorbance of the reference, the integrated photoluminescence intensity of the reference, and the refractive index of the solvents.

### Entrapment Efficiency of Dyes in Nanoparticles

Entrapment efficiency of dyes was measured via a reported protocol. The absorbance of PdTPBP or perylene in dichloromethane (A₀) before synthesis of the TTA-UCNP was measured. The dichloromethane was added to extract the PdTPBP and the perylene from the final TTA-UCNP product to attain absorbance (A) for PdTPBP or perylene in the TTA-UCNP. Then the entrapment efficiency of PdTPBP or perylene was calculated according to Equation (2) that has been used similarly in the literature. (Huang, et al. 2016 J. Am. Chem. Soc. 138, 14586-14591.)

$$Entrapment efficiency ( \% ) = ( A ) / ( {A}_{0} ) \times 100 \qquad ( 2 )$$

### TTA Upconversion Measurement of the TTA-UCNP in PBS Buffer

A continuous diode-pumped solid-state laser (650 nm) was used as the excitation source for the upconversion. For the upconversion measurements, the TTA-UCNP was irradiated by 650 nm laser under different conditions (in the absence of glucose and GOX, in the presence of glucose and GOX, in the presence of glucose or GOX alone). The upconversion emission spectra of TTA-UCNP were recorded with a HORIBA spectrofluorometer after the upconversion emission intensities of the TTA-UCNP were stabilized. The Φ_{UC} of the TTA-UCNP in PBS buffer was calculated according to above-mentioned equation. The TTA upconversion brightness is then calculated according to Equation (3) that has been used similarly in the literature (Wu, et al. 2012 J. Org. Chem. 77, 5305-5312)

$$η = {Φ}_{uc} \times ε \qquad ( 3 )$$

where η is the TTA upconversion brightness; Φ_{UC} is the TTA upconversion quantm yield of TTA-UCNP; ε is the molar extinction absorption coefficient of PdTPBP.

### Triplet-Triplet Energy Transfer (TTET) Efficiency Measurements

The PdTPBP-to-perylene triplet-triplet energy transfer quantum yield in TTA-UCNP was calculated from the measurements of the PdTPBP phosphorescence in the presence of perylene (I, TTA-UCNP) and in the absence of the perylene (I₀, PdTPBP NP), using Equation (4) that has been used in the literature. (Mattiello, et al. 2016 Adv. Funct. Mater. 26, 8447-8454.)

$${Φ}_{( TTET )} = 1 - I / {I}_{0} \qquad ( 4 )$$

### Calculation of the Detection Limit of Glucose

The detection limit of glucose was calculated based on a reported method. (Peng, et al. 2009 Sens. Actuators B Chem. 136, 80-85.) A linear relation exists between the half response time (y, t_{½}) and the concentration of glucose in In-In coordination (FIG. 3c). This linear relationship is fitted as in Equation (5)

$$y = - 0.628 x + 3.27 \qquad ( 5 )$$

Based on the theory of Martins and Naes, the detection limit can be derived from the signal processing performance, as in Equation (6) described below (Martins, T. Naes, Multivariate Calibration, Wiley & Sons, New York, 1998)

$${V}_{{x}^{2}} = \sum {( {y}_{i} - y )}^{2} \qquad ( 6 )$$

where yᵢ is the average value from the calculation and y is the measured data points.

The background of root-mean-square (rms) rmsₙₒᵢₛₑ is calculated using Equation (7)

$${rms}_{noise} = \sqrt[2]{\frac{{V}_{{x}^{2}}}{N}} \qquad ( 7 )$$

Where N is the number of data points (N=3) used for the average value.

Then the detection limit of the glucose is calculated to be 0.06 mg mL⁻¹ using Equation (8).

$$The detection limit = 3 \times {rms}_{noise} / ( slope of the In - In coordination line ) \qquad ( 8 )$$

### Measurement of Invertase Activity

First, the half response time of TTA-UCNP (1 mg mL⁻¹) in conjunction with GOX (10 μg mL⁻¹) was measured at different concentrations of glucose (0, 0.1, 0.2, 0.3, 0.4, 0.5, 0.7, 1.1, 1.5, 2.0 mg mL⁻¹) to attain the standard curve in deionized water. The results were shown in FIG. 4b. Next, sucrose (100 mg mL⁻¹) and different concentrations of invertase were incubated at room temperature for 5 min. After that, the invertase was deactivated in boiling water (100° C.) for 10 min. When the solution further cooled down to room temperature (25° C.), TTA-UCNP (1 mg mL⁻¹) and GOX (10 μg mL⁻¹) were added. The half response times of the TTA-upconversion were then measured. According to the standard curve (FIG. 4b), the glucose concentration in the solution can be calculated and deduced. Since in the reaction a mole of sucrose is hydrolyzed to produce a mole glucose and a mole fructose, the amount of sucrose is equal to that of glucose. Therefore, the amount of hydrolyzed sucrose can be calculated using the amount of glucose. Then the actual invertase activity (U) can be calculated using Equation (9) that has been used in a commercial protocol. (The commercialized products for invertase activity assay kit (sensitive): https://www.abcam.com/invertase-activity-assay-kit-colorimetric-ab197005.html.)

$$Invertase activity ( U ) = Amount of sucrose ( µmol ) / 5 min . \qquad ( 9 )$$

### Calculation of the Detection Limit of Invertase

The process for the calculation of detection limit of invertase is similar with the above-mentioned calculation of the detection limit of glucose. A linear relation exists between the half response time (y, t_{½}) and the activity of invertase (x, unit) in In-In coordination (FIG. 4c). This linear relation is fitted as Equation (10):

$$y = - 0.625 x + 3.28 \qquad ( 10 )$$

Then using the above-mentioned equations for glucose detection limit, the invertase detection limit is calculated to be 0.01 unit.

Applicant's disclosure is described herein in preferred embodiments with reference to the Figures, in which like numbers represent the same or similar elements. Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

The described features, structures, or characteristics of Applicant's disclosure may be combined in any suitable manner in one or more embodiments. In the description, herein, numerous specific details are recited to provide a thorough understanding of embodiments of the invention. One skilled in the relevant art will recognize, however, that Applicant's composition and/or method may be practiced without one or more of the specific details, or with other methods, components, materials, and so forth. In other instances, well-known structures, materials, or operations are not shown or described in detail to avoid obscuring aspects of the disclosure.

In this specification and the appended claims, the singular forms “a,” “an,” and “the” include plural reference, unless the context clearly dictates otherwise.

Unless defined otherwise, all technical and scientific terms used herein have the same meaning as commonly understood by one of ordinary skill in the art. Although any methods and materials similar or equivalent to those described herein can also be used in the practice or testing of the present disclosure, the preferred methods and materials are now described. Methods recited herein may be carried out in any order that is logically possible, in addition to a particular order disclosed.

### INCORPORATION BY REFERENCE

References and citations to other documents, such as patents, patent applications, patent publications, journals, books, papers, web contents, have been made in this disclosure. All such documents are hereby incorporated herein by reference in their entirety for all purposes. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

### EQUIVALENTS

The representative examples are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples and the references to the scientific and patent literature included herein. The examples contain important additional information, exemplification and guidance that can be adapted to the practice of this invention in its various embodiments and equivalents thereof.

1. An upconversion nanoparticle comprising an organic photosensitizer and an organic annihilator pair encapsulated in a polymeric material, wherein upon excitation the organic photosensitizer-annihilator pair is capable of undergoing triplet-triplet annihilation upconversion to generate emission having a shorter wavelength than that of the excitation.

2. The upconversion nanoparticle of claim 1, wherein the organic photosensitizer is selected from the group consisting of: iridium complexes, metal-porphyrin complexes, metal-benzoporphyrin complexes, boron-dipyrromethene (BODIPY) or derivatives thereof; and the organic annihilator is selected from the group consisting of: coumarin or derivatives thereof, anthracene derivatives, perylene or derivatives thereof.

3. The upconversion nanoparticle of claim 2, wherein the organic photosensitizer is a metal-porphyrin complex.

4. The upconversion nanoparticle of claim 2, wherein the organic photosensitizer is a metal-benzoporphyrin complex.

5. The upconversion nanoparticle of claim 1, wherein the organic annihilator is perylene or a derivative thereof.

6. The upconversion nanoparticle of claim 5, wherein the organic photosensitizer is palladium (II) meso-tetraphenyl-tetrabenzoporphyrin (PdTPBP) and the organic annihilator is perylene.

7. The upconversion nanoparticle of claim 1, wherein the polymeric material is an amphiphilic polymer.

8. The upconversion nanoparticle of claim 7, wherein the amphiphilic polymer is selected from dodecane substituted PAA and octadecane substituted PAA.

9. The upconversion nanoparticle of claim 1, wherein the polymeric material is polyacrylic acid substituted octadecylamine (PAA-OA).

10. The upconversion nanoparticle of claim 1, having a particle size in the range of about 50 nm to about 200 nm.

11. The upconversion nanoparticle of claim 1, having a particle size less than about 50 nm.

12. The upconversion nanoparticle of claim 1, being water dispersible.

13. An aqueous composition comprising a plurality of the upconversion nanoparticle of claim 1.

14. The aqueous composition of claim 13, further comprising glucose.

15. The aqueous composition of claim 13, further comprising glucose oxidase (GOX).

16. The aqueous composition of claim 13, further comprising glucose and glucose oxidase (GOX).

17. A sensor device comprising a upconversion nanoparticle of claim 1.

18. A method for detecting glucose, comprising:
providing an aqueous composition comprising a upconversion nanoparticle of claim 1 and glucose oxidase (GOX);
measuring a rate of triplet-triplet annihilation upconversion;
adding to the aqueous composition a test sample;
measuring again the rate of triplet-triplet annihilation upconversion; and
analyzing the presence and/or concentration of glucose based on measurements with and without the test sample.

19-22. (canceled)

23. A method for measuring activity of an enzyme related to glucose metabolism, comprising:
providing an aqueous composition comprising a upconversion nanoparticle of claim 1 and glucose;
measuring a rate of triplet-triplet annihilation upconversion;
adding to the aqueous composition a test sample;
measuring again the rate of triplet-triplet annihilation upconversion; and
analyzing enzymatic activity based on measurements with and without the test sample.

24-28. (canceled)
